(19) United States
(12) Patent Application Publication
(10) Pub. No.: US 2019/0069875 A1
(43) Pub. Date: Mar. 7, 2019

(54) ULTRASOUND SYSTEM FOR CEREBRAL BLOOD FLOW IMAGING AND MICROBUBBLE-ENHANCED BLOOD CLOT LYSIS

(71) Applicant: KONINKLIJKE PHILIPS N.V., Eindhoven (NL)

(72) Inventors: Jeffry Earl Powers, Bainbridge Island, WA (US); Tracy C. Brechbiel, Lake Stevens, WA (US); William Tao Shi, Wakefield, MA (US); Ralf Seip, Carmel, NY (US)

(21) Appl. No.: 15/767,241

(22) PCT Filed: Oct. 11, 2016
(86) PCT No.: PCT/EP2016/074298

§ 371 Date: Apr. 10, 2018

Related U.S. Application Data
(60) Provisional application No. 62/241,408, filed on Oct. 14, 2015.

(51) Int. Cl. A61B 8/08; A61B 8/06; A61B 8/00

(52) U.S. Cl. CPC A61B 8/0816; A61B 8/06; A61N 2007/0078; A61B 8/4254; A61B 8/4209; A61B 8/4488

(57) ABSTRACT

An ultrasonic diagnostic imaging system is described which can diagnose, treat, or monitor the cranial vasculature for obstructions such as blood clots causing ischemic stroke. The system has a headset which maintains two transducer arrays in contact with acoustic windows through the temporal bones on opposite sides of the head. The clinician is aided in properly positioning the arrays over the best acoustic windows through the bone by a signal produced by one of the arrays in response to transmission through the cranium by the other array, which passes through the temporal bones on both sides of the head. The amplitude of this through- transmission signal is detected and displayed to the clinician, either qualitatively or quantitatively, as the arrays are positioned.

This disclosure relates to medical diagnostic ultrasound systems and, in particular, to ultrasound systems which perform imaging and therapy in the cranium of a patient.

Ischemic stroke is one of the most debilitating disorders known to medicine. The blockage of the flow of blood to the brain can rapidly result in paralysis or death. Attempts to achieve recanalization through thrombolytic drug therapy such as treatment with tissue plasminogen activator (tPA) have been reported to cause symptomatic intracerebral hemorrhage in a number of cases. Advances in the diagnosis and treatment of this crippling affliction are the subject of continuing medical research.

Transcranial Doppler ultrasound has been developed for use in monitoring and diagnosing stroke. A headset device manufactured by Spencer

Technologies of Seattle, Wash., USA holds two transducers against the side of the skull, one on each temporal bone just in front of the ear. The transducers transmit ultrasonic waves through the temporal bone and the returning echo signals are Doppler processed and the phase shift information reproduced at audible frequencies. The audible Doppler identifies the presence or absence of blood flow inside the cranium as the clinician listens for characteristic sounds of blood flow velocities of specific arteries. The technique can also be augmented with a spectral Doppler display of the phase shift information, providing information on flow velocities inside the cranium. However, since there is no information concerning the anatomy inside the skull, the clinician must attempt to make a diagnosis on the basis of this limited information.

U.S. Pat. No. 8,211,023 (Swan et al.) describes a diagnostic ultrasound system and method which enable a clinician to transcranially visualize a region of the cerebral vasculature where blood clots may be present. Either two dimensional or three dimensional imaging may be employed. The imaging of the vasculature is preferably enhanced by the administration of contrast microbubbles. If the flow conditions of the vasculature indicate the presence of a partial or complete occlusion, a focused or pencil beam is directed to the location of the blockage to break up the clot by the vibrations and/or rupturing of the microbubbles. In some instances the ruptured microbubbles may also release an encapsulated thrombolytic drug. The patent also describes monitoring the cranial vasculature by ultrasonic imaging for changes which are indicative of the recurrence of an occlusion so that medical aid can be alerted to the recurrent condition.

In these procedures the ultrasound is administered transcranially, through the bones of the skull. These bones attenuate the ultrasonic energy passing through them. It has been found that the relatively thin temporal bones provide some of the most effective acoustic windows through the skull for ultrasound. However the location of the best acoustic window through the temporal bones is not always apparent. Bone is highly attenuating to ultrasound and human skull bone windows, in particular the temporal bone windows, vary in size, thickness, and even location. It is desirable to provide a way for the clinician to find the best acoustic window through the temporal bones so that the transducer may be positioned appropriately for transmission through this window.

In some aspects, the present disclose includes an ultrasound system for cranial diagnosis, monitoring and/or therapy. The system can include first and second arrays of ultrasonic transducer elements, a transducer array headset configured to maintain the ultrasonic transducer arrays in contact with acoustic windows on opposite sides of a head of a subject, a detector that is coupled to an element of the second array and configured to produce a signal in response to the reception of ultrasound by the element of the second array in response to a transmission of ultrasound by the first array, and a display that is coupled to the detector and configured to produce an indicator of quality for one or both of the acoustic windows. In certain aspects, the first and second arrays can include two dimensional arrays of transducer elements. An element of the second array to which the detector is coupled can include a central element of the second array. The element of the second array to which the detector is coupled can include a plurality of commonly operated central elements of the second array.

In certain aspects, the first array can be configured to operate as an imaging array and the element of the second array is configured to operate as a receiving element for ultrasound transmitted by the first array. The first array can be configured to operate as an imaging array and the element of the second array configured to operate as a receiving element for ultrasound transmitted by the first array during positioning of at least one of the ultrasonic transducer arrays in contact with an acoustic window. The second array can be configured to operate as an imaging array and an element of the first array is configured to operate as a receiving element for ultrasound transmitted by the second array.

The system can also include an image processor configured to produce an ultrasound image in response to scanning of an image field by the first array and to modulate at least a portion of the ultrasound image in response to the signal produced by the detector. The modulated portion of the ultrasound image can be modulated in brightness.

In some aspects, the indicator can include an indicator bar. The indicator can include a numerical indicator representing a numerical value in response to the signal produced by the detector. The system can include a graphics processor coupled to the detector. The display can be responsive to the graphics processor and configured to display a dynamic indicator of variation of a detector signal from the detector. The dynamic indicator can include a bar indicator. The dynamic indicator can include a scrolling line.

In certain aspects, the detector can include an amplifier producing a signal with an amplitude that is proportionate to the signal produced by the element of the second array. The system can further include an A/D converter coupled to the element of the second array which produces a digital representation of the amplitude of the signal produced by the element of the second array.

In some aspects, the present disclosure can include ultrasound systems that are configured to carry out the methods disclosed herein. For instance, the present disclosure can include an ultrasound system having instructions thereon, which when executed, cause the system to produce a signal in response to the reception of ultrasound by an element of a second array of transducer elements in response to a transmission of ultrasound by a first array of transducer elements, and display an indicator of quality for one or two acoustic windows that correspond to opposite sides of a patient's head on which the first and second array are mounted.

In the drawings:

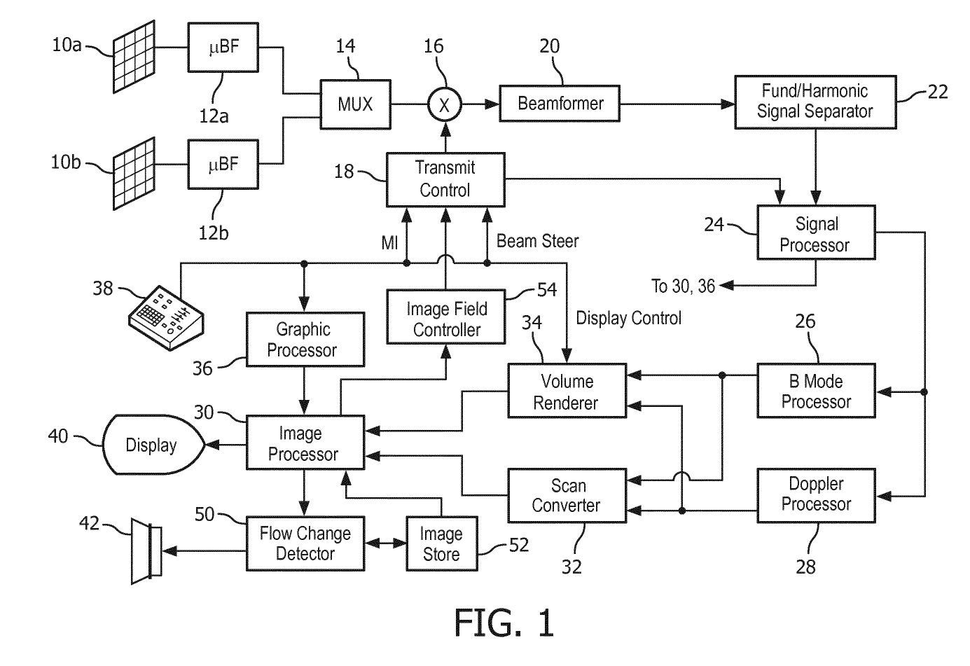

FIG. 1 illustrates in block diagram form an ultrasonic diagnostic imaging system constructed in accordance with the principles of the present disclosure.

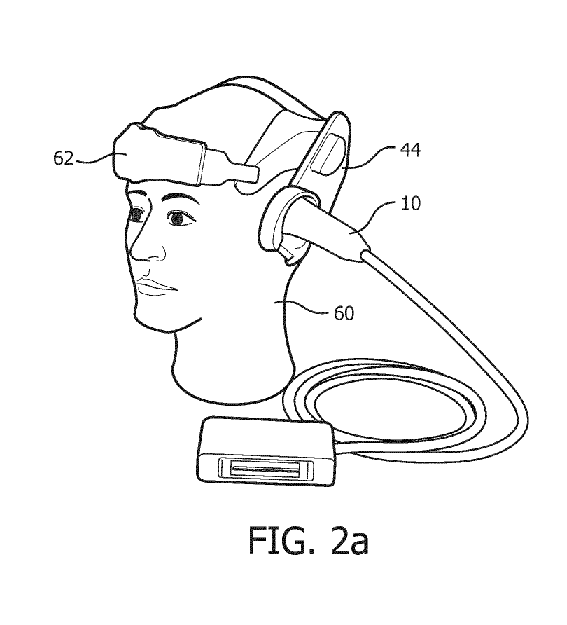

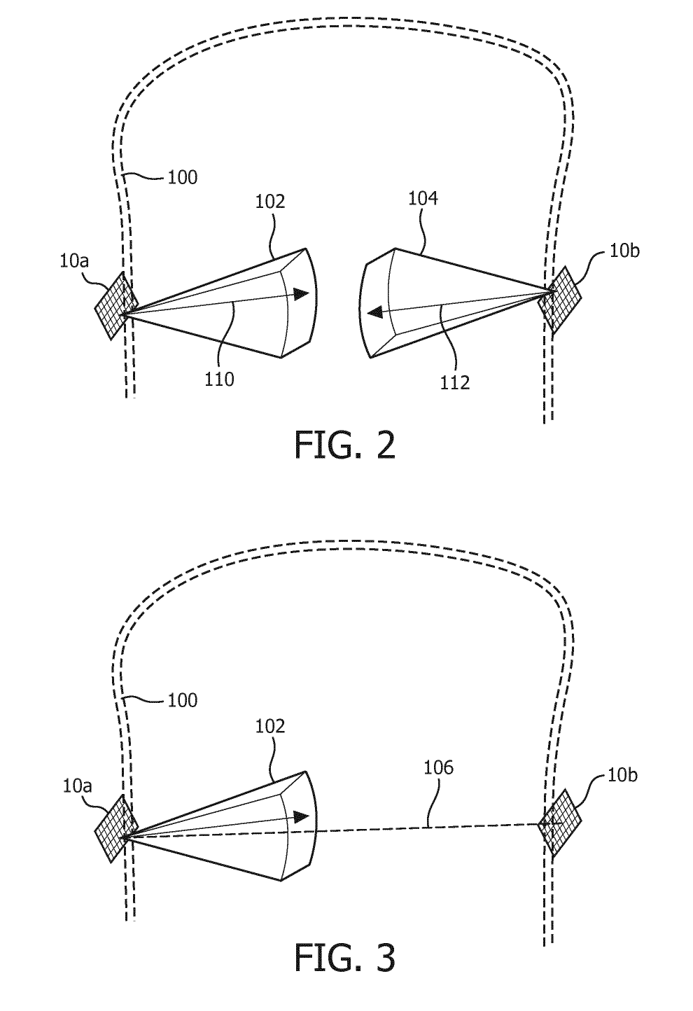

FIG. 2 illustrates regions of the cranium being imaged from transducer arrays located over the temporal bone on either side of the head.

FIG. 2a illustrates a cranial headset suitable for holding transducer arrays in acoustic contact with the temporal bone regions of the head.

FIG. 3 illustrates the reception of ultrasound from one transcranial transducer array by a contralateral transducer array in accordance with the principles of the present disclosure.

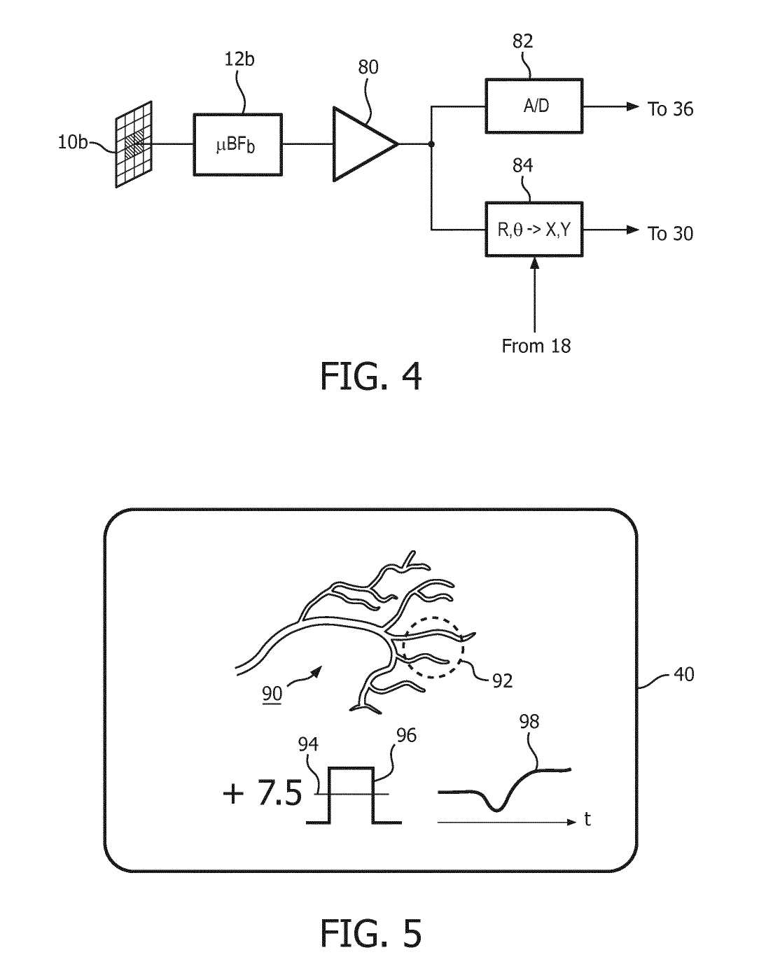

FIG. 4 illustrates the signal path from the contralateral transducer array of FIG. 3 in an implementation of the present disclosure.

FIG. 5 illustrates a display of beam intensity through the cranium in accordance with the present disclosure.

In accordance with the principles of the present disclosure, two matrix array transducers can be located on either side of the head over the general location of the temporal bones. While one transducer is transmitting ultrasound into the cranium, the other contralateral transducer is receiving the transmitted ultrasound with one or more of its transducer elements. These elements thus receive the ultrasound transmitted through the cranium/temporal bones and brain. The amplitude of the received signals is monitored while the position of one or both of the transducer arrays is adjusted until the greatest signal amplitude is received, thereby enabling the positioning of the transducers over the most effective acoustic windows.

Referring first to FIG. 1, an ultrasound system constructed in accordance with the principles of the present disclosure is shown in block diagram form. In some aspects, the ultrasound systems include various structures standard in computers, such as microprocessors, integrated circuits, (e.g., FPGAs), memory, hard drives, etc. Two transducer arrays 10a and 10b are provided for transmitting ultrasonic waves and receiving echo information. In this example the arrays shown are two dimensional arrays of transducer elements (matrix arrays) capable of providing 3D image information although an implementation of the present disclosure may also use two dimensional arrays of transducer element which produce 2D (planar) images. In some embodiments, the array of transducer elements can be coupled to a system beamformer depending on the element count. For higher element counts, the transducer arrays can be coupled to microbeamformers 12a and 12b which control transmission and reception of signals by the array elements. Microbeamformers are also capable of at least partial beamforming of the signals received by groups or “patches” of transducer elements as described in U.S. Pat. No. 5,997,479 (Savord et al.), U.S. Pat. No. 6,013,032 (Savord), and U.S. Pat. No. 6,623,432 (Powers et al.) Signals are routed to and from the microbeamformers by a multiplexer 14 by time-interleaving signals. The multiplexer is coupled to a transmit/receive (T/R) switch 16 which switches between transmission and reception and protects the main beamformer 20 from high energy transmit signals. The transmission of ultrasonic beams from the transducer arrays 10a and 10b under control of the microbeamformers 12a and 12b is directed by the transmit controller 18 coupled to the T/R switch, which received input from the user's operation of the user interface or control panel 38 and controls the steering direction and focusing of beams to and from the array transducer in accordance with system control settings. The transmit controller can include configurable hardware, such as a microprocessor, or integrated circuit or other hardware chip-based device.

The partially beamformed signals produced by the microbeamformers 12a, 12b are coupled to a main beamformer 20 where partially beamformed signals from the individual patches of elements are combined into a fully beamformed signal. For example, the main beamformer 20 may have 128 channels, each of which receives a partially beamformed signal from a patch of 12 transducer elements. In this way the signals received by over 1500 transducer elements of a two dimensional array can contribute efficiently to a single beamformed signal. In an example where, for example, 128 transducer elements are used in the array, then the elements can be coupled directly to main beamformer 20 without use of any microbeamformers.

The beamformed signals are coupled to a fundamental/harmonic signal separator 22. The separator 22 acts to separate linear and nonlinear signals so as to enable the identification of the strongly nonlinear echo signals returned from microbubbles. The separator 22 may operate in a variety of ways such as by bandpass filtering the received signals in fundamental frequency and harmonic frequency bands, or by a process known as pulse inversion harmonic separation. A suitable fundamental/harmonic signal separator is shown and described in international patent publication WO 2005/074805 (Bruce et al.) The separated signals are coupled to a signal processor 24 where they may undergo additional enhancement such as speckle removal, signal compounding, and noise elimination.

The processed signals are coupled to a B mode processor 26 and a Doppler processor 28. The B mode processor 26 employs amplitude detection for the imaging of structures in the body such as muscle, tissue, and blood cells. B mode images of structure of the body may be formed in either the harmonic mode or the fundamental mode. Tissues in the body and microbubbles both return both types of signals and the harmonic returns of microbubbles enable microbubbles to be clearly segmented in an image. The Doppler processor processes temporally distinct signals from moving tissue and blood flow for the detection of motion of substances in the image field including microbubbles. The structural and motion signals produced by these processors are coupled to a scan converter 32 and a volume renderer 34, which produce image data of tissue structure, flow, or a combined image of both characteristics. The scan converter will convert echo signals with polar coordinates into image signals of the desired image format such as a sector image in Cartesian coordinates. The volume renderer 34 will convert a 3D data set into a projected 3D image as viewed from a given reference point as described in U.S. Pat. No. 6,530,885 (Entrekin et al.) As described therein, when the reference point of the rendering is changed the 3D image can appear to rotate in what is known as kinetic parallax. This image manipulation is controlled by the user as indicated by the Display Control line between the user interface 38 and the volume renderer 34. Also described is the representation of a 3D volume by planar images of different image planes, a technique known as multiplanar reformatting. The volume renderer 34 can operate on image data in either rectilinear or polar coordinates as described in U.S. Pat. No. 6,723,050 (Dow et al.) The 2D or 3D images are coupled from the scan converter and volume renderer to an image processor 30 for further enhancement, buffering and temporary storage for display on a display 40.

A graphics processor 36 is also coupled to the image processor 30 which generates graphic overlays for displaying with the ultrasound images. These graphic overlays can contain standard identifying information such as patient name, date and time of the image, imaging parameters, and the like, and can also produce a graphic overlay of a beam vector steered by the user as described below. For this purpose the graphics processor received input from the user interface 38. The user interface is also coupled to the transmit controller 18 to control the generation of ultrasound signals from the transducer arrays 10a and 10b and hence the images produced by and therapy applied by the transducer arrays. The transmit parameters controlled in response to user adjustment include the MI (Mechanical Index) which controls the peak intensity of the transmitted waves, which is related to cavitational effects of the ultrasound, steering of the transmitted beams for image positioning and/or positioning (steering) of a therapy beam.

The transducer arrays 10a and 10b transmit ultrasonic waves into the cranium of a patient from opposite sides of the head, although other locations may also or alternately be employed such as the front of the head or the sub-occipital acoustic window at the back of the skull. The sides of the head of most patients advantageously provide suitable acoustic windows for transcranial ultrasound at the temporal bones around and above the ears on either side of the head. In order to transmit and receive echoes through these acoustic windows the transducer arrays must be in good acoustic contact at these locations which may be done by holding the transducer arrays against the head with a headset. For instance, FIG. 2a shows a headset 62 for two matrix array probes 10 mounted on the head 60 of a mannequin. The sides of the head of most patients advantageously provide suitable acoustic windows for transcranial ultrasound at the temporal bones around and in front of the ears on either side of the head. In order to transmit and receive echoes through these acoustic windows the transducer arrays must be in good acoustic contact at these locations which may be done by holding the transducer arrays against the head with the headset 62. A headset may have a snap-on deformable acoustic standoff 44 which allows the transducer array to be manipulated by its conformal contact surface and aimed at the arteries within the brain while maintaining acoustic contact against the temporal window. The illustrated probe 10 is curved by bending the probe handle by 90°, which makes the probe more stable when attached to the headset 62, as its center of gravity is closer to the head and headset. The acoustic coupling objective is facilitated by integrating a mating spherical surface into the probe handle, which allows the probe to pivot in the headset 62 until it is strongly and tightly coupled to the temporal window of the patient.

FIG. 2 illustrates the volumetric image fields 102, 104 scanned by matrix array transducers 10a and 10b when acoustically coupled to scan through the skull 100. A clinician can image the cranial vasculature in these volumetric image fields and steer the pyramidal image fields in different directions to search for obstructions to the cranial blood flow. At each position of the image field 102, 104 the clinician can look for obstructions of the blood flow in the real time images on the display, or can capture (freeze) an image or map of the cranial vasculature. When the vascular map is acquired and held statically, the image can undergo enhanced processing (e.g., compounding, signal averaging) to improve the resolution or scale of the image, and can be manipulated on the screen and examined carefully at different points and from different views in a precise search for blood vessel occlusions. In this way the clinician can diagnose for stenoses. If the clinician examines a vascular map and finds no evidence of obstruction in the blood flow paths, the clinician can steer the image field to another region of the cranium and examine the vascular map of another image field. The clinician can use the Doppler data of the vascular map or the spectral Doppler function of the ultrasound system to take flow velocity measurements at specific points in the cranial vasculature, then use the report generation capabilities of the ultrasound system to record the measurements and prepare a report of his diagnosis.

If the clinician discovers a stenosis, therapy can be applied by agitating or breaking microbubbles at the site of the stenosis in an effort to dissolve the blood clot. The clinician activates the “therapy” mode of the ultrasound system, and a graphic 110, 112 appears in the image field 102, 104, depicting the vector path of a therapeutic ultrasound beam. The therapeutic ultrasound beam is manipulated by a control on the user interface 38 until the vector graphic 110 or 112 is focused at the site of the blockage. The therapeutic beam can be a tightly focused, convergent beam or a beam with a relatively long focal length known as a pencil beam. The energy produced for the therapeutic beam can be in excess of the ultrasound levels permitted for diagnostic ultrasound, in which case the microbubbles at the site of the blood clot will be sharply broken. The energy of the resulting microbubble ruptures will strongly agitate the blood clot, tending to break up the clot and dissolve it in the bloodstream. However in some instances insonification of the microbubbles at diagnostic energy levels may be sufficient to dissolve the clot. Rather than breaking in a single event, the microbubbles may be vibrated and oscillated, and the energy from such extended oscillation prior to dissolution of the microbubbles can be sufficient to break up the clot.

In the depiction of FIG. 4, each image field 102, 104 is seen to extend almost halfway across the cranium, which is a balance between the size of the image field and the acoustic penetration and attenuation which may be expected through the bone at the acoustic window. For some patients, low attenuation effects may enable an image field to extend fully across the cranium, allowing the clinician to examine the vascular structure near the skull bone on the opposite side of the cranium. By alternately examining image fields of both transducer arrays, the vasculature across the full cranium may be effectively examined. It is possible to acquire extended image fields which cover the same central region of the cranium but imaged from opposite sides of the head. These images can be correlated and compounded together, forming a fused image that may reveal additional characteristics of the brain. The therapeutic beam can be transmitted from both sides of the head, enabling breakup of a clot at both sides of the clot. Rather than be limited to reflective ultrasound imaging, through-transmission imaging can be performed by transmitting ultrasound from one transducer array and receiving the remaining unabsorbed ultrasonic energy at the other transducer array, which may reveal yet other characteristics of the brain tissue.

It is common in the case of stroke that the affliction will not manifest itself in a single episode, but in repeated episodes as a blood clot or obstruction in the heart, lungs, or blood vessel breaks up gradually, releasing small clots which successively make their way to the vascular system of the brain over time. Thus, a patient who survives an initial stroke event, may be at risk for other events in the near future. Accordingly, it is desirable to monitor these patients for some time after an initial stroke event so that recurrences can be treated immediately. The ultrasound system of FIG. 1 may be used for the monitoring of stroke victims for recurrent events. In a monitoring mode, successive images of the vasculature are stored in an image store 52, and temporally different images of the vascular map are compared to detect changes in flow of the vasculature by operation of flow change detector 50. The flow change detector operates by comparing the identical nature of the temporally different images, similar to the image data correlation techniques used to identify motion by image processing as described in U.S. Pat. No. 6,442,289 (Olsson et al.) As long as successive images appear substantially the same in their flow characteristics, e.g., there is no localized change in the flow characteristics of a particular section of the vasculature and no section of the vasculature has ceased to return a Doppler signal indicating the continuation of flow, the flow change detector 50 will continue its monitoring of the vasculature with no change. But if a comparison by the flow change detector indicates a change in blood flow, such as the loss of Doppler-detected flow in a section of the vascular network, an audible alarm 42 is activated. Medical assistance can then be brought immediately to the patient. In addition, the images stored in the image store at the time of the detected flow change can be examined by medical personnel to discern exactly where in the vasculature the detected obstruction occurred. Therapy can then be directed specifically to the site of the obstruction without the need to closely examine a series of vascular maps.

While the monitoring implementation can be performed with 2D (planar) imaging, it is preferred that 3D imaging be used so that a larger volumetric region can be monitored. Monitoring can be performed with only one transducer array, but a greater number of arrays likewise provide monitoring of a larger region of the cranium.

All of the foregoing diagnostic, therapy, and monitoring modes require that the ultrasound transducer be acoustically coupled to the head to send and receive ultrasound through a good acoustic window, so that high quality images may be formed and effective therapy applied. In accordance with the principles of the present disclosure, the ultrasound system of FIG. 1 enables the clinician to find the best acoustic window through the temporal bones when positioning the transducers. This is done by using the transducer on one side of the head to monitor the amount of ultrasonic energy delivered by the other transducer while positioning the transducers against the head. This is illustrated in FIG. 3, in which matrix array 10a is transmitting ultrasound beams to scan an image region 102. The contralateral transducer 10b has one or more of its transducer elements operating as a receiver. The central element of the array 10b can be used as the receiving element, but in this example the four central elements are coupled together, causing them to commonly operate as a single element with a larger receiving surface and hence greater sensitivity. Alternatively, a number of elements can be hard-wired together, but using a main beamformer or combination of a microbeamformer and a main beamformer to connect the transducers only during transducer placement enables them to be returned to their normal imaging and therapy operations during diagnosis, treatment, and monitoring. A dashed line 106 indicates the path of one beam from the transmitting array 10a to the central receiving elements of array 10b. The signals received by the central receiving elements of array 10b are amplitude-detected and the detected signal amplitude is displayed to the clinician as a guide in transducer placement. For instance, moving transducer 10a around over the temporal bones will cause the transmitted beams to pass through a different region of bone. This can cause the signals received by the central elements of array 10b to increase or decrease in amplitude as the ultrasound now may pass through thicker or thinner or more or less dense bone. The clinician observes the changing amplitude of the received signals on a display until the acoustic window which is best transmissive of ultrasound is found. After positioning transducer 10a the clinician can similarly move transducer 10b until its best acoustic window is found. The process is then repeated with transducer 10b operating as the transmitting array and the central elements of transducer 10a operating as the receiving elements. At the conclusion of this process the two transducer arrays are positioned over the most efficient acoustic windows of the temporal bones, and patient diagnosis and treatment can begin.

The construction of the positioning amplitude signal path in the ultrasound system of FIG. 1 is illustrated in FIG. 4. The four center elements of the matrix array 10b are coupled to microbeamformer 12b where their signals are combined and coupled to an amplifier and amplitude detector 80. The detected signal is converted to a digital value by an A/D converted 82 and the digital value of the detected signal amplitude is coupled to the graphics processor 36 where it is displayed on the image display 40 as shown in FIG. 5. The detected signal is also applied to a scan converter 84, which also receives from the transmit controller 18 the current beam direction in which a beam is being transmitted by the contralateral array 10a. The scan converter converts the R,θ beam direction to the Cartesian x,y coordinates used by scan converter 32 to form a scan converted image for display. The detected positioning amplitude signal and the current beam coordinates are coupled to the image processor 30, which modulates the intensity of the displayed image at the coordinates of the transmit beam. In FIG. 1, the detector 80, the A/D converter 82 and the scan converter 84 are implemented as part of the signal processor 24.

The way this modulation affects the displayed image is illustrated in FIG. 5, which shows the display screen 40 during the transducer positioning process. In the upper part of the screen is the ultrasound image, which in this example is the vascular map 90 acquired by scanning the image field 102. In a typical scan sequence beams are sequentially transmitted along parallel rows through the image region from top to bottom until echoes have been acquired from the entire region. The echoes are processed to form a B mode or, in this example, a flow image 90 of the cranial vasculature. When the imaging array 10a is aimed in the general direction of array 10b, as it will naturally when both arrays are located on opposite sides of the head, one or several of the scanned beams will travel in the direction of dashed line 106 in FIG. 3 and be received by the central elements of the contralateral array 10b. These beam locations in the ultrasound image are modulated to be displayed more brightly in proportion to the amplitude of the detected positioning signal. In the example of FIG. 5, the beams that scanned the region of the image outlined by dashed circle 92 were also detected by the contralateral array elements and are displayed more brightly than the rest of the vascular map 90. The clinician can thereby gain a sense of the respective aiming of the two arrays toward each other. As the clinician moves transducer 10a around over the temporal bone area, the brightened area of the image will also move and change in brightness, becoming brighter when a good acoustic window is encountered and less bright when thicker or denser bone is encountered, giving the clinician a good sense of the relative transducer positioning.

The display of FIG. 5 illustrates several other ways to produce a dynamic indicator of the positioning signal amplitude to the clinician. One way is with a numerical indicator, shown by the number +7.5. This can be a unitless or normalized value, as it is only necessary to present a number which varies one way as the acoustic window improves, and the other way as the acoustic window deteriorates. In the illustrated example the number +7.5 will increase with a stronger positioning signal as a more favorable acoustic window is found, and will decrease as the acoustic window becomes worse and the positioning signal amplitude decreases. The example in the center of the display is an indicator bar 96 which increases in height as the acoustic window improves, and decreases as the acoustic window degrades. A line 94 marks the initial bar height at the start of the positioning process as a point of reference for the clinician. To the right of the display is a line 98 which continually scrolls across the screen during the positioning process. When the acoustic window improves and a greater amplitude positioning signal is produced, the scrolling line 98 increases in height. When the acoustic window becomes poorer, the scrolling line decreases in height. When the transducers are stationary, the scrolling line assumes a straight line shape. In the illustrated example, the line curvature indicates that the acoustic window got worse with the initial adjustment of transducer position, then improved to a window even better than at the start with further repositioning of a transducer.

Other approaches for displaying the transducer positioning signal amplitude to a user will readily occur to those skilled in the art. An implementation of the present disclosure can use 1D arrays, 1.5D arrays or 2D arrays, with two dimensional arrays being preferred for their ability to scan a volumetric image region. The transducer element(s) used to detect the positioning signal are preferably elements of the imaging/therapy array, but can alternatively be implemented as separate, dedicated positioning signal sensor elements.

It will be understood that each block of the block diagram illustrations, and combinations of blocks in the block diagram illustrations, as well any portion of the systems and methods disclosed herein, can be implemented by computer program instructions. These program instructions may be provided to a processor to produce a machine, such that the instructions, which execute on the processor, create means for implementing the actions specified in the block diagram block or blocks or described for the systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the disclosure. The computer program instructions can be stored on any suitable computer-readable hardware medium including, but not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device.

1. An ultrasound system for cranial diagnosis, monitoring and/or therapy comprising:
a first and a second array of transducer elements;
a transducer array headset configured to maintain the first and second arrays in contact with first and second acoustic windows on a first and second side of a patient's head, respectively;
a detector that is coupled to a transducer element of the second array and configured to produce a signal in response to the reception of ultrasound by the transducer element of the second array in response to reception of a transmission of ultrasound by the first array;
a graphics processor coupled to the detector, the graphics processor configured to produce a graphical indicator of signal amplitude, based at least in part on an amplitude of the tansmission of ultrasoung by the first array received by the second array; and
a display that is coupled to the graphics processor and configured to display the graphical indicator.

2. The ultrasound system of claim 1, wherein the first and second arrays further comprise two dimensional arrays of transducer elements; and
wherein the transducer element of the second array to which the detector is coupled further comprises a central transducer element of the second array.

3. The ultrasound system of claim 2, wherein the transducer element of the second array to which the detector is coupled further comprises a plurality of commonly operated central transducer elements of the second array.

4. The ultrasound system of claim 1, wherein the first array is configured to operate as an imaging array and the transducer element of the second array is configured to operate as a receiving transducer element for ultrasound transmitted by the first array.

5. The ultrasound system of claim 4, wherein the first array is configured to operate as an imaging array and the transducer element of the second array configured to operate as a receiving transducer element for ultrasound transmitted by the first array during positioning of at least one of the ultrasonic transducer arrays in contact with an acoustic window.

6. The ultrasound system of claim 4, wherein the second array is configured to operate as an imaging array and a transducer element of the first array is configured to operate as a receiving transducer element for ultrasound transmitted by the second array.

7. The ultrasound system of claim 1, wherein the indicator comprises an indicator bar.

8. The ultrasound system of claim 7, further comprising an image processor configured to produce an ultrasound image in response to scanning of an image field by the first array and to modulate at least a portion of the ultrasound image in response to the signal produced by the detector.

9. The ultrasound system of claim 8, wherein the modulated portion of the ultrasound image is modulated in brightness to indicate the amplitude of the transmission of ultrasound by the first array received by the second array.

10. The ultrasound system of claim 1, wherein the graphical indicator further comprises a numerical indicator representing a numerical value in response to the signal produced by the detector.

11. The ultrasound system of claim 7,
wherein the graphical indicator comprises a dynamic indicator of variation of the amplitude of the transmission of ultrasound by the first array received by the second array.

12. The ultrasound system of claim 11, wherein the dynamic indicator comprises a bar indicator.

13. The ultrasound system of claim 11, wherein the dynamic indicator further comprises a scrolling line.

14. The ultrasound system of claim 1, wherein the detector comprises an amplifier producing a signal with an amplitude that is proportionate to the signal produced by the transducer element of the second array.

15. The ultrasound system of claim 1, further comprising an A/D converter coupled to the transducer element of the second array which produces a digital representation of an amplitude of the signal produced by the transducer element of the second array.
